(19) United States
(12) Patent Application Publication
(10) Pub. No.: US 2017/0119704 A1
(43) Pub. Date: May 4, 2017

(54) TREATMENT OF COGNITIVE IMPAIRMENT

(71) Applicant: Ovid Therapeutics Inc., New York, NY (US)

(72) Inventor: Matthew DURING, Weston, CT (US)

(21) Appl. No.: 15/292,660

(22) Filed: Oct. 13, 2016

Related U.S. Application Data
(60) Provisional application No. 62/240,740, filed on Oct. 13, 2015.
(60) Provisional application No. 62/259,818, filed on Nov. 25, 2015.

(51) Int. Cl. A61K 31/15

(52) U.S. Cl. CPC A61K 31/15

(57) ABSTRACT

Provided are methods of treating cognitive impairment and/or psychosis and/or movement disorders by administering a pharmaceutical composition of eplivanserin or a pharmaceutically acceptable salt thereof to a subject in need thereof.

### CROSS-REFERENCE TO RELATED APPLICATIONS

This application claims benefit of and priority to U.S. Provisional Application No. 62/240,740, filed Oct. 13, 2015, and U.S. Provisional Application No. 62/259,818, filed Nov. 25, 2015, the entire contents of each of which are incorporated herein by reference.

### TECHNICAL FIELD

Treatment of cognitive impairment associated with brain disorders.

### BACKGROUND

Eplivanserin ((1Z,2E)-1-(2-fluorophenyl)-3-(4-hydroxyphenyl)-prop-2-en-1-one-O-(2-dimethylaminoethyl)-oxime; SR46349; or SR 46349B) is an antagonist of the serotonin 5HT_{2A} receptor and is described, e.g., in EP 373998 and U.S. Pat. No. 5,166,416. Eplivanserin was an experimental drug in clinical development by Sanofi Aventis for the treatment of insomnia and has been disclosed for other sleep indications. For example, U.S. Pat. No. 6,143,792 describes treatments for sleep apnea and U.S. Pat. No. 6,576,970 describes treatments for snoring and upper airway resistance syndrome. However, development of eplivanserin was discontinued in December 2009 and it has not been approved for any indication.

Cognitive impairment is a condition associated with a large number of brain disorders. Brain disorders can have many causes, e.g., degenerative conditions, heredity, trauma, infection, malnutrition and others. For example, cognitive impairment can be associated with aging and/or neurodegenerative diseases such as Alzheimer's disease, Parkinson's disease, Huntington's disease, Amyotrophic Lateral Sclerosis (ALS), psychosis, Parkinson's disease psychosis, Alzheimer's disease psychosis, Lewy-body dementia, prionic neurodegenerative disorders such as Creutzfeld-Jacob disease and kuru disease, corticobasal degeneration, frontotemporal lobar degeneration, multiple sclerosis, normal pressure hydrocephalus, organic chronic brain syndrome, Pick's disease, progressive supranuclear palsy, or senile dementia. Cognitive impairment may also have a congenital basis, e.g., Prader-Willi syndrome, Down Syndrome, Fragile X Syndrome, Angelman syndrome and autism spectrum disorder. Cognitive impairment may also be associated with trauma to the brain, such as that caused by chronic subdural hematoma, concussion, stroke, intracerebral hemorrhage, or with other injury to the brain, such as that caused by infection (e.g., encephalitis, meningitis, septicemia) or drug intoxication or abuse. Cognitive impairment may also be associated with other conditions which impair or otherwise affect normal functioning of the central nervous system, including sleep deprivation, psychiatric disorders such as anxiety disorders, dissociative disorders, mood disorders, schizophrenia, treatment with psychiatric medications, treatment with dopamine agonists and somatoform and factitious disorders; it may also be associated with conditions of the peripheral nervous system, such as chronic pain. In some cases, the cause of a cognitive impairment may be unknown or uncertain.

Cognitive impairment can be manifest in many ways, e.g., deficits in learning and/or memory including, but not limited to, attention, information acquisition, information processing, working memory, short-term memory, long-term memory, anterograde memory, retrograde memory, memory retrieval, discrimination learning, decision-making, language retrieval, inhibitory response control, attentional set-shifting, delayed reinforcement learning, reversal learning, the temporal integration of voluntary behavior, and expressing an interest in one's surroundings and self-care. Cognitive impairment may be characterized by progressive loss of memory, cognition, reasoning, executive functioning, planning, judgment and emotional stability

Although many advances have been made, treatments for cognitive impairment associated with brain disorders remain largely inadequate. For diseases such as Huntington's disease, Parkinson's disease, Amyotrophic Lateral Sclerosis, Alzheimer's disease, Prader-Willi syndrome, Lewy body dementia and others, treatments may be limited or unavailable. There is a need for additional therapeutic options for treating cognitive impairment associated with brain disorders.

### SUMMARY

Methods of treating cognitive impairment by administering a pharmaceutical composition of eplivanserin or a pharmaceutically acceptable salt thereof to a patient in need of such treatment are provided. In embodiments, cognitive impairment is characterized by a deficit in mental activities associated with thinking, reasoning, learning, memory and/or sensory processing. In embodiments, cognitive impairment can be agnosia, amnesia, aphasia, apraxia, delirium, dementia and/or a learning disorder.

In embodiments, cognitive impairment is associated with neurodegenerative disease, brain trauma, psychiatric disorders or chronic pain. In embodiments, cognitive impairment is associated with a condition selected from the group consisting of AIDS dementia complex, Binswanger's disease, Lewy body dementia, frontotemporal dementia, mild cognitive impairment, multi-infarct dementia, Pick's disease, semantic dementia, Alzheimer's disease, Parkinson's disease, Huntington's disease, Amyotrophic Lateral Sclerosis (ALS), Creutzfeld-Jacob disease, Kuru disease, corticobasal degeneration, frontotemporal lobar degeneration, multiple sclerosis, normal pressure hydrocephalus, organic chronic brain syndrome, Pick's disease, progressive supranuclear palsy, senile dementia, vascular dementia, autism spectrum disorder, Down Syndrome, Fragile X Syndrome, Angelman syndrome, Prader-Willi syndrome, Rett syndrome, childhood disintegrative disorder, attention deficit disorder, attention deficit hyperactivity disorder, obsessive-compulsive disorder, brain trauma, infection, drug intoxication, drug abuse, treatment with psychiatric medications, treatment with dopamine agonists, sleep deprivation, anxiety disorders, dissociative disorders, mood disorders, schizophrenia, somatoform and factitious disorders and chronic pain.

In embodiments, eplivanserin or a pharmaceutically acceptable salt of eplivanserin is administered from one to three times a day. In embodiments, from 1 mg to 50 mg of eplivanserin or a pharmaceutically acceptable salt of eplivanserin is administered within a 24-hour period.

### DETAILED DESCRIPTION

Eplivanserin or a pharmaceutically acceptable salt thereof is used to treat a patient suffering from one or more types of cognitive impairment. The term “cognitive impairment,” as used herein, means any condition characterized by a deficit in mental activities associated with thinking, reasoning, learning, memory and/or sensory processing. Examples of such conditions include agnosia, amnesia, aphasia, apraxia, delirium, dementia and learning disorders.

Cognitive impairment may be associated with a large number of conditions. Examples are aging and/or neurodegenerative diseases such as Alzheimer's disease, Parkinson's disease, Huntington's disease, Amyotrophic Lateral Sclerosis (ALS), Lewy-body dementia, prionic neurodegenerative disorders such as Creutzfeld-Jacob disease and kuru disease, corticobasal degeneration, frontotemporal lobar degeneration, multiple sclerosis, normal pressure hydrocephalus, organic chronic brain syndrome, Pick's disease, progressive supranuclear palsy, or senile dementia. Cognitive impairment may also be associated with developmental disorders, e.g., Down Syndrome, Fragile X Syndrome, Angelman syndrome, Prader-Willi syndrome and autism spectrum disorder. Autism spectrum disorder encompasses previous diagnoses of autistic disorder, Asperger's disorder, childhood disintegrative disorder and pervasive developmental disorder not otherwise specified (PDD-NOS) according to the American Psychiatric Association's Diagnostic and Statistical Manual of Mental Disorders, 5th edition (DSM-5) (APA 2013).

Cognitive impairment may also be associated with trauma to the brain, such as that caused by chronic subdural hematoma, concussion, stroke, intracerebral hemorrhage, or with other injury to the brain, such as that caused by infection (e.g., encephalitis, meningitis, septicemia) or drug intoxication or abuse. Cognitive impairment may also be associated with other conditions which impair normal functioning of the central nervous system, including sleep deprivation, psychiatric disorders such as anxiety disorders, dissociative disorders, mood disorders, obsessive compulsive disorders, schizophrenia, and somatoform and factitious disorders. Cognitive impairment may also be associated with conditions of the peripheral nervous system, such as chronic pain. Cognitive impairment may also be associated with administration of medications such as those used to treat psychiatric disorders and medications such as those used to treat Parkinson's disease, e.g., dopamine agonists. In some cases, the cause of a cognitive impairment may be unknown or uncertain.

Criteria regarding learning disorders are provided in the DSM-5 that considers specific learning disabilities to be a type of neurodevelopmental disorder that impedes the ability to learn or use specific academic skills (e.g., reading, writing, or arithmetic), which are the foundation for other learning.

Cognitive impairment may be measured against normal cognitive function, which refers to the normal physiologic activity of the brain, including, but not limited to, one or more of the following: mental stability, memory/recall abilities, problem solving abilities, reasoning abilities, thinking abilities, judging abilities, ability to discriminate or make choices, capacity for learning, ease of learning, perception, intuition, attention, and awareness, as measured by any criteria suitable in the art.

Cognitive impairment also includes deficits in mental activities that are mild or that otherwise do not significantly interfere with daily life. Mild cognitive impairment (MCI) is an example of such a condition. A patient with mild cognitive impairment may display symptoms of dementia (e.g., difficulties with language or memory) but the severity of these symptoms is such that a diagnosis of dementia may not be appropriate.

One skilled in the art will appreciate that there are numerous human and animal models that may be used to evaluate and compare the relative safety and efficacy of eplivanserin for the treatment of cognitive impairment. In humans, cognitive function may be measured, for example and without limitation, by the clinical global impression of change scale (CGI); the Mini Mental State Exam (MMSE) (aka the Folstein Test); the Neuropsychiatric Inventory (NPI); the Clinical Dementia Rating Scale (CDR); the Cambridge Neuropsychological Test Automated Battery (CANTAB), the Sandoz Clinical Assessment-Geriatric (SCAG) scale, the Benton Visual Retention Test (BVRT), Montreal Cognitive Assessment (MoCA) or Digit Symbol Substitution Test (DSST).

In animal model systems, cognitive function may be measured in various conventional ways known in the art, including using a Morris Water Navigation Task, Barnes maze, radial arm maze task, T maze and the like. Other tests known in the art may also be used to assess cognitive function, such as novel object recognition and odor recognition tasks.

Cognitive function may also be measured using imaging techniques such as Positron Emission Tomography (PET), functional magnetic resonance imaging (fMRI), Single Photon Emission Computed Tomography (SPECT), or any other imaging technique that allows one to measure brain function. In animals, cognitive function may also be measured with electrophysiological techniques.

Accordingly, eplivanserin or a pharmaceutically acceptable salt thereof is used to treat a subject suffering from cognitive impairment. The subject may be an animal, e.g., mammal, e.g., human, etc. As used herein, the terms “treat”, “treatment” or “treating” encompass any manner in which the symptoms or pathology of a condition, disorder or disease are ameliorated or otherwise beneficially altered. In embodiments, “treat”, “treatment” or “treating” can refer to inhibiting a disease or condition, e.g., arresting or reducing its development or at least one clinical or subclinical symptom thereof. In embodiments, “treat”, “treatment” or “treating” can refer to relieving the disease or condition, e.g., causing regression of the disease or condition or at least one of its clinical or subclinical symptoms. In embodiments, “treating cognitive impairment” means ameliorating, beneficially altering and/or providing relief from one or more of the symptoms of cognitive impairment. The benefit to a subject being treated may be statistically significant, mathematically significant, or at least perceptible to the subject and/or the physician.

Methods described herein also include treatment of psychosis by administering eplivanserin or a pharmaceutically acceptable salt thereof to a patient in need thereof. Psychosis is a mental disorder that may be characterized by thinking and emotions that are so impaired. In some instances the impairment from psychosis may be severe such that the patient losses contact with reality. Patients experiencing psychosis may be characterized by false thoughts (delusions) and/or see or hear things that are not there (hallucinations). These are referred to as “positive” symptoms of psychosis. Psychosis may also include “negative” symptoms such as, e.g., loss of motivation and social withdrawal.

Symptoms of psychosis that may be treated with eplivanserin or a pharmaceutically acceptable salt thereof may include difficulty concentrating, depressed mood, sleep changes—sleeping too much or not enough, anxiety, suspiciousness, withdrawal from family and friends and/or ongoing unusual thoughts and beliefs. Additional symptoms of psychosis that may be treated with eplivanserin or a pharmaceutically acceptable salt thereof may include delusions, hallucinations, disorganized speech (e.g., switching topics erratically), depression, anxiety, suicidal thoughts or actions, and/or overall difficulty functioning

In embodiments, methods herein include treating Parkinson's disease psychosis by administering eplivanserin or a pharmaceutically acceptable salt thereof to a patient in need thereof. Methods herein also include treating Alzheimer's disease psychosis by administering eplivanserin or a pharmaceutically acceptable salt thereof to a patient in need thereof. In embodiments, methods include treating Lewy-body dementia by administering eplivanserin or a pharmaceutically acceptable salt thereof.

In embodiments, methods herein include treating movement disorders by administering to a patient in need thereof a pharmaceutical composition including eplivanserin or a pharmaceutically acceptable salt thereof. In embodiments, methods herein include alleviating a movement abnormality associated with a movement disorder including administering to a patient in need thereof a pharmaceutical composition comprising an effective amount of eplivanserin or a pharmaceutically acceptable salt thereof. In embodiments, methods of treating a dyskinetic symptom in a patient with a movement disorder, include administering to the patient a pharmaceutical composition comprising an effective amount of eplivanserin or a pharmaceutically acceptable salt thereof. In embodiments, methods of controlling tics and vocal utterances of Tourette's Disorder include administering to the patient a pharmaceutical composition comprising an effective amount of eplivanserin or a pharmaceutically acceptable salt thereof. In embodiments, methods of treating obsessions and compulsions in patients with Obsessive-Compulsive Disorder include administering to the patient a pharmaceutical composition comprising an effective amount of eplivanserin or a pharmaceutically acceptable salt thereof. In embodiments, methods for the acute and maintenance treatment of Obsessive Compulsive Disorder include administering to the patient a pharmaceutical composition comprising an effective amount of eplivanserin or a pharmaceutically acceptable salt thereof. In embodiments, methods for the suppression of motor and phonic tics in patients with Tourette's Disorder include administering to the patient a pharmaceutical composition comprising an effective amount of eplivanserin or a pharmaceutically acceptable salt thereof. In embodiments, movement disorders may include, e.g., Tourette's syndrome, tic disorders, obsessive-compulsive disorder, Attention deficit hyperactivity disorder, Parkinson's disease, Huntington's chorea, Wilson disease, akathisia, or dyskinesia. In embodiments, the movement disorder is tardive dyskinesia. In embodiments, the movement disorder is a chronic motor or vocal tic disorder.

In embodiments, the terms “effective amount” or “therapeutically effective amount” refer to an amount of a compound, material, composition, medicament, or other material that is effective to achieve a particular pharmacological and/or physiologic effect. Such results include, but are not limited to, one or more of the following: enhancing cognitive function, increasing daytime activity, improving learning (either the rate or ease of learning), improving attention, improving social behavior, and/or improving cerebrovascular function. In embodiments, effective amount refers to an amount which may be suitable to prevent a decline in any one or more of the above qualities, or, in embodiments, to improve any one or more of the above qualities, for example, cognitive function or performance, learning rate or ability, problem solving ability, attention span and ability to focus on a task or problem, social behavior, and the like. In embodiments, an effective amount may be suitable to reduce either the extent or rate of decline in a subject's cognitive skills or functioning, and/or the effective amount may be suitable to delay the onset of such decline. Such effectiveness may be achieved, for example, by administering compositions described herein to an individual or to a population. In embodiments, the reduction, or delay of such a decline, or the improvement in an individual or population can be relative to a cohort, e.g., a control subject or a cohort population that has not received the treatment, or been administered the composition or medicament.

Eplivanserin can exist in the form of two isomers, according to the Z or E configuration

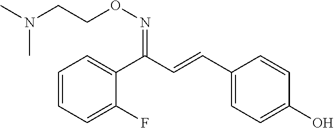 [SMILES: CN(C)CCO/N=C(/C=C/c1ccc(O)cc1)c1ccccc1F]

In embodiments, eplivanserin or a pharmaceutically acceptable salt thereof may include a racemic mixture, as well as compositions including each isomer individually. The compositions and methods described herein may provide reduced dosing frequency, reduced adverse events and/or increased efficacy compared to a mixture of eplivanserin isomers. In embodiments, compositions and methods that include each isomer individually may provide reduced dosing frequency, reduced adverse events and/or increased efficacy compared to the minor isomer. Thus, in embodiments, contemplated herein are compositions and methods of treatment that provide the Z isomer of eplivanserin or a pharmaceutically acceptable salt thereof that is substantially free of the E isomer of eplivanserin. In embodiments, the methods and compositions described herein include the E isomer of eplivanserin or a pharmaceutically acceptable salt thereof substantially free of the Z isomer of eplivanserin. By “substantially free” it is meant that the composition includes less than 50% of the minor isomer of eplivanserin. In embodiments, the compositions and methods described herein may include less than about, e.g., 25%, 15%, 10%, 8%, 5%, 3%, 2%, or less than 1% of the minor E isomer of eplivanserin.

In embodiments, the methods and compositions described herein include the Z isomer of eplivanserin or a pharmaceutically acceptable salt thereof. In embodiments, the compositions include more than, e.g., about 75%, about 85%, about 90%, about 95% or about 98% Z isomer of eplivanserin. In embodiments, the compositions include between, e.g., about 50% to about 75%, about 75% to about 100%, about 85% to about 100%, about 90% to about 100%, or about 95% to about 100% Z isomer of eplivanserin.

In embodiments, eplivanserin or pharmaceutically acceptable salts may include the hemifumarate salt. Eplivanserin is well absorbed (>70%). Conventional dosage, between 1 and 10 mg, leads to a maximal plasma concentration that is reached between 2 and 6 hours; the half-life of eplivanserin or pharmaceutically acceptable salts or esters thereof is relatively long, with an average value of 50 hours.

In embodiments, the methods and compositions described herein include between from about 0.01 to about 50 mg eplivanserin or pharmaceutically acceptable salt thereof. In embodiments, the methods and compositions described herein include between from, e.g., about 0.01 to about 25 mg, about 0.01 to about 15 mg, about 0.01 to about 10 mg, or about 0.01 to about 5 mg eplivanserin or pharmaceutically acceptable salt thereof.

Embodiments described herein can include doses, e.g., in the range of about 0.1 to 20 mg, 1 to 30 mg, 5 to 25 mg, 7.5 to 15 mg, or 10 to 25 mg, 2.5 to 5 mg, or 1 to 5 mg, or 1 to 10 mg, or 1 to 20 mg, or 1 to 25 mg, with doses of, e.g., about 1 mg, 1.5 mg, 1.25 mg, and 1.75 mg, 2.0 mg, 5 mg, 10 mg, 15 mg, 20 mg, 2.5 mg, 3.0 mg, 3.5 mg, 4.0 mg, 4.5 mg, 0.5 mg, 0.25 mg, and 0.75 mg being examples.

In embodiments, pharmaceutical compositions may include, e.g., about 0.1 mg to 5 mg, 0.1 mg to 10 mg, 0.1 mg to 15 mg, 0.5 mg to 5 mg, 0.5 mg to 1 mg, 0.5 to 15 mg, 1 mg to 10 mg, 1 mg to 20 mg, 1 mg to 15 mg, 1.5 mg to 15 mg, 1.5 mg to 20 mg, 1.5 mg to 10 mg, 1.5 mg to 5 mg, 2 mg to 20 mg, 2 mg to 15 mg, 2 mg to 10 mg, 2.5 mg to 20 mg, 2.5 mg to 10 mg, 2.5 mg to 15 mg, 3 mg to 25 mg, 3 mg to 20 mg, 3 mg to 15 mg, or 3 mg to 10 mg eplivanserin or a pharmaceutically acceptable salt thereof.

In embodiments, pharmaceutical compositions may include 0.1 mg, 0.25 mg, 0.5 mg, 0.75 mg, 1 mg, 2.0 mg, 2.5 mg, 3 mg, 4 mg, 5 mg, 7 mg, 7.5 mg, 10 mg, 12.5 mg, 15 mg, 17.5 mg, 20 mg eplivanserin or a pharmaceutically acceptable salt thereof or amounts that are multiples of such doses. Typically, dosages may be administered once, twice, or three times daily, or every other day to a human.

In embodiments, the dosage is 0.01-100 mg/kg, 0.5-50 mg/kg, 0.5-10 mg/kg or 25-50 mg/kg once or twice daily. For example, in embodiments, the dosage is 0.5 mg/kg, 1 mg/kg, 5 mg/kg, 7.5 mg/kg, or 10 mg/kg once or twice daily. In embodiments, a subject is administered a total daily dose of 1 mg to 50 mg of eplivanserin or a pharmaceutically acceptable salt thereof once, twice (e.g., b.i.d.), or three times (e.g., t.i.d.) daily. In embodiments, the total amount administered to a subject in 24-hour period is 1 mg to 50 mg. In embodiments, the subject may be started at a low dose and the dosage is escalated.

Suitable dosage forms for eplivanserin or pharmaceutically acceptable salts or esters thereof include, but are not limited to oral forms, such as tablets, hard or soft gelatin capsules, powders, granules and oral solutions or suspensions, as well as the sublingual, buccal, intratracheal, intraocular, intranasal forms, forms adapted to inhalation, topical, transdermal, sub-cutaneous, intramuscular or intravenous delivery, rectal forms such as suppositories, and implants for release of medication. For topical application, eplivanserin or pharmaceutically acceptable salts or esters thereof may be used as creams, gels, ointments or lotions. Suitable dosage forms for eplivanserin or pharmaceutically acceptable salts or esters can be formulated for immediate release, sustained release or delayed release. Sustained release formulations release the active ingredient over an extended period of time. In embodiments, the sustained release of the active ingredient may be substantially constant over time. In embodiments, sustained release of the active ingredient may release specified doses at timed intervals. Delayed release formulations typically provide a period of delay before the active ingredient is released.

The terms “about” or “approximately” as used herein mean within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, a range up to 10%, a range up to 5%, and/or a range up to 1% of a given value. Alternatively, particularly with respect to biological systems or processes, the term can mean within an order of magnitude, e.g., within 5-fold, or within 2-fold, of a value. “About” and “approximately” are used interchangeably herein.

“Pharmaceutically acceptable” refers to molecular entities and compositions that are “generally regarded as safe”—e.g., that are physiologically tolerable and do not typically produce an allergic or similar untoward reaction when administered to a human. In embodiments, this term refers to molecular entities and compositions approved by a regulatory agency of the federal or a state government, as the GRAS list under section 204(s) and 409 of the Federal Food, Drug and Cosmetic Act, that is subject to premarket review and approval by the FDA or similar lists, the U.S. Pharmacopeia or another generally recognized pharmacopeia for use in animals, and more particularly in humans.

“Pharmaceutically acceptable salt” refers to derivatives of eplivanserin wherein the compound is modified by making acid or base salts thereof. Examples of pharmaceutically acceptable salts include but are not limited to mineral or organic acid salts of basic residues such as amines, and alkali or organic salts of acidic residues such as carboxylic acids. Pharmaceutically acceptable salts include conventional non-toxic salts or quaternary ammonium salts of the parent compound formed, for example, from non-toxic inorganic or organic acids. Such conventional non-toxic salts include those derived from inorganic acids such as hydrochloric, hydrobromic, sulfuric, sulfamic, phosphoric, and nitric acids; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, tolunesulfonic, naphthalenesulfonic, methanesulfonic, ethane disulfonic, and oxalic salts. In embodiments, eplivanserin or pharmaceutically acceptable salts may include a hemifumarate salt. The pharmaceutically acceptable salts of eplivanserin can be synthesized from the parent compound, which contains a basic or acidic moiety, by conventional chemical methods.

Pharmaceutically acceptable dosage forms may include adjuvants or excipients such as, but not limited to, diluents, binders, lubricants, disintegrants, fillers, stabilizers, glidants and coating compositions. Pharmaceutically acceptable adjuvants or excipients and methods of compounding utilizing pharmaceutically acceptable adjuvants or excipients are well known in the art.

The following prophetic examples are provided as an aid for understanding and appreciating the present disclosure. The examples are not intended to limit the disclosure herein to any particular embodiment.

### EXAMPLES

### Example 1

This prospective study may be used to evaluate safety and efficacy in Parkinson's disease and psychosis. An objective of this prospective study will be to determine if eplivanserin will ameliorate psychosis in patients with Parkinson's disease. Another objective of this prospective study will be to demonstrate that eplivanserin will not worsen parkinsonism in, patients with Parkinson's disease and psychosis. This study may be a multi-center, randomized, placebo-controlled, double blind trial in which patients meeting entrance criteria will be randomly assigned to receive placebo or active drug.

Enrolled patients may have a clinical diagnosis of idiopathic Parkinson's disease, defined as the presence of at least three of the cardinal features of the disease including: rest tremor, rigidity, bradykinesia and/or akinesia, postural balance abnormalities, in the absence of alternative explanations or atypical features. Psychosis may be defined by the presence of visual and/or auditory hallucinations, with or without delusions, of at least four weeks duration. Psychosis may be assessed with standard scales such as the Neuropsychiatric Inventory (NPI) or Schedule for Assessment of Positive Symptoms (SAPS).

The criteria for including a patient may be determined using the NPI and a total assessment score which may be defined as Hallucinations (Frequency×Severity) and Delusions (Frequency×Severity) of, e.g., 4 or greater. The study may include a 4-week design such that patients will take eplivanserin daily starting on Day 1. Dose escalations can occur on y Days 8 and 15, and patients will receive a stable daily dosage from Day 16 until Day 28. Single step dose reductions may be allowed during that period for adverse events or intolerance. The major response variable will be motoric tolerability. Secondary response variables will be efficacy against psychosis and safety.

### Example 2

Prospective study to evaluate the safety and efficacy of eplivanserin compared to placebo in patients with Parkinson's disease psychosis. This prospective study will be a multi-Center, placebo-controlled, double blind trial to examine the safety and efficacy of the treatment of psychosis in Parkinson's Disease. The study will measure antipsychotic efficacy that may be defined as a decrease in the severity and/or frequency of hallucinations and/or delusions. This is measured as the change from baseline (Day 1) to the end of study, or at intermediate periods, using the Scale for the Assessment of Positive Symptoms—Hallucinations and Delusions scales (SAPS-H+D) score. The possible total score is 0 to 100 and a negative change in score indicates improvement. Motor symptoms may also be measured using the change from baseline (Day 1) to the end of study, or at intermediate periods, using the combined score of the Unified Parkinson's Disease Rating Scale (UPDRS) Part II (Activities of Daily Living) and Part III (Motor Examination) using the per-protocol (PP) analysis set. The possible total score is 0 to 160 and a negative change in score indicates improvement.

### Example 3

The next-day cognitive performance after repeated administration of eplivanserin may be measured and compared to placebo and/or a hypnotic drug, such as flurazepam as the active control.

### Study Design:

A prospective single center study will employ a two-part, double-blind, randomized, placebo- and active-controlled, 4-way crossover design. Eligibility criteria will include certification of good health; 18-70 years of age, and written informed consent.

Part A (the first part of the study): participants are first administered either one evening dose of flurazepam or matching placebo, and after a 21-day washout period, participants are administered the other treatment.

Part B: participants will receive either an evening dose of eplivanserin or matching placebo for 21 days.

After this phase of the study, participants are then administered the reverse treatment sequence.

Next-day morning (7:05-7:25 am), cognitive performance will be assessed using 6 serial tests:

Bond-Lader Visual Analog Scale (VAS)

Immediate recall of Word list

Compensatory Tracking Task (CTT)

Critical Flicker Fusion (CFF) test

Choice Reaction Time (CRT)

Delayed Recall of Word Lists

Seven primary endpoints will be derived from the 6 following psychometric tests:

Bond and Lader Visual Analogue Scale (VAS), showing the alertness;

Immediate recall of Word List, showing the number of correct words;

Compensatory Tracking test (CTT), showing both the mean deviation and the mean response time;

Critical Flicker Fusion (CFF) threshold;

Choice Reaction Time (CRT), showing the total reaction time

Delayed recall of Word List, showing the number of correct words.

For parts A and B, these 7 primary endpoints (raw data) will be first analyzed simultaneously using a multivariate mixed effect model (global analysis). Then for each primary endpoint (individual analysis), the difference will be compared with placebo derived from the model. For each study part, percent change from placebo will be computed.

In addition, cognitive function may be assessed using one or more of the following: the clinical global impression of change scale (CGI); the Mini Mental State Exam (MMSE) (aka the Folstein Test); the Neuropsychiatric Inventory (NPI); the Clinical Dementia Rating Scale (CDR); the Cambridge Neuropsychological Test Automated Battery (CANTAB), the Sandoz Clinical Assessment-Geriatric (SCAG) scale, the Benton Visual Retention Test (BVRT), Montreal Cognitive Assessment (MoCA) or Digit Symbol Substitution Test (DSST).

### Example 4

### Prospective Study to Determine Efficacy and/or Safety of Eplivanserin Reducing Total Tic Severity in Children and Adolescents with Tourette's Disorder

Tourette's Disorder is a neuropsychiatric condition that is characterized by the appearance of tics that can be simple or complex in nature. A tic is a sudden, rapid, recurrent, non-rhythmic, stereotyped motor movement or vocalization. The goal of this prospective trial is to obtain efficacy, safety, and tolerability data of eplivanserin in children and adolescents with Tourette's Disorder. The trial may have an 8-week double-blind treatment period after a pretreatment (screening/washout phase), and the subjects should be followed-up with for 1 month after the last treatment. The daily dosage regimen of eplivanserin administered to children and adolescent may be evaluated using a Primary Outcome Measures of Change From Baseline to Week 8 in Yale Global Tic Severity Scale (YGTSS) Total Tic Score (TTS). The YGTSS is a semi-structured clinical interview designed to measure current (time frame of the past 1 week) tic severity. This scale consists of a tic inventory, with 5 separate rating scales to rate the severity of symptoms, and an impairment ranking. Ratings are made along 5 different dimensions on a scale of 0 to 5 for motor and vocal tics each, including number, frequency, intensity, complexity, and interference. Summation of these 10 scores (i.e., 0-50) provides a TTS that was the primary outcome measure in this trial. The YGTSS ranking of impairment score rated on a 50-point scale anchored from 0 (no impairment) to 50 (severe impairment) to assess impairment experienced in areas of self-esteem, family life, social acceptance, and school scores. This is a fully validated scale in adults and has become a standard instrument for the evaluation of the severity of TD in children.

Secondary Outcome Measures may include Change in Clinical Global Impressions Scale-Tourette's Syndrome (CGI-TS) Score at Week 8. To assess CGI-TS severity, the rater or physician may answer questions, such as: “Considering your total clinical experience with this particular population, how mentally ill is the patient at this time?” However, the evaluation of illness should be limited to manifestations of TD only. Response choices included: 0=not assessed; 1=normal, not at all ill; 2=borderline mentally ill; 3=mildly ill; 4=moderately ill; 5=markedly ill; 6=severely ill; and 7=among the most extremely ill patients. The change score was obtained from CGI-TS improvement scale assessment: 0=not assessed, 1=very much improved, 2=much improved, 3=minimally improved, 4=no change, 5=minimally worse, 6=much worse, and 7=very much worse.

### Example 5

### Prospective Study to Assess the Safety and/or Efficacy of Eplivanserin for the Treatment of Obsessive Compulsive Behavior

The goal of this prospective trial is to obtain efficacy, safety, and tolerability data of eplivanserin for the potential treatment of Obsessive Compulsive Behavior (OCD). The daily dosage regimen of eplivanserin administered may be evaluated using a Primary Outcome Measures of OCD severity by examining the change from baseline in the Yale-Brown Obsessive-Compulsive Scale (Y-BOCS) ratings of OCD severity at 1 day following administration. The Yale-Brown Obsessive Compulsive Scale (Y-BOCS) assesses obsessive and compulsive symptom severity. Obsessions are rated on a scale from 0-20 and compulsions are rated on a scale of 0-20, for a total scale of 0-40. Scores on the obsessions scale and scores on the compulsions scale are summed to obtain the total score. The higher the score, the more severe the OCD.

It should be understood that the examples and embodiments provided herein are exemplary examples and embodiments. Those skilled in the art will envision various modifications of the examples and embodiments that are consistent with the scope of the disclosure herein. Such modifications are intended to be encompassed by the claims.

What is claimed is:

1. A method for treating cognitive impairment comprising administering eplivanserin or a pharmaceutically acceptable salt of eplivanserin to a subject in need of such treatment.

2. The method of claim 1, wherein the cognitive impairment is characterized by a deficit in mental activities associated with thinking, reasoning, learning, memory and/or sensory processing.

3. The method of claim 2, wherein the cognitive impairment is selected from the group consisting of agnosia, amnesia, aphasia, apraxia, delirium, dementia and a learning disorder.

4. The method of claim 1, wherein the cognitive impairment is associated with a condition selected from the group consisting of AIDS dementia complex, Binswanger's disease, Lewy-body dementia, frontotemporal dementia, mild cognitive impairment, multi-infarct dementia, Pick's disease, semantic dementia, Alzheimer's disease, Parkinson's disease, Huntington's disease, Amyotrophic Lateral Sclerosis (ALS), Creutzfeld-Jacob disease, Kuru disease, corticobasal degeneration, frontotemporal lobar degeneration, multiple sclerosis, normal pressure hydrocephalus, organic chronic brain syndrome, Pick's disease, progressive supranuclear palsy, senile dementia, vascular dementia, autism spectrum disorder, Down Syndrome, Fragile X Syndrome, Angelman syndrome, Prader-Willi syndrome, Rett syndrome, childhood disintegrative disorder, attention deficit disorder, attention deficit hyperactivity disorder, obsessive-compulsive disorder, brain trauma, infection, drug intoxication, drug abuse, sleep deprivation, anxiety disorders, dissociative disorders, mood disorders, schizophrenia, treatment with psychiatric medications, treatment with dopamine agonists, somatoform and factitious disorders and chronic pain.

5. The method of claim 1, wherein the cognitive impairment is associated with neurodegenerative disease, brain trauma, psychiatric disorders or chronic pain.

6. The method of claim 5, wherein brain trauma is selected from the group consisting of chronic subdural hematoma, concussion, intracerebral hemorrhage, encephalitis, meningitis, septicemia, drug intoxication and drug abuse.

7. The method of claim 5, wherein the psychiatric disorders are selected from the group consisting of anxiety disorders, dissociative disorders, mood disorders, schizophrenia, and somatoform and factitious disorders.

8. The method of claim 5, wherein the neurodegenerative disease is Huntington's disease.

9. The method of claim 5, wherein the neurodegenerative disease is Parkinson's disease.

10. The method of claim 5, wherein the neurodegenerative disease is Amyotrophic lateral sclerosis.

11. The method of claim 5, wherein the neurodegenerative disease is Alzheimer's disease.

12. The method of claim 5, wherein the neurodegenerative disease is Lewy-body dementia.

13. The method of claim 1, wherein eplivanserin or a pharmaceutically acceptable salt of eplivanserin is administered from one to three times a day.

14. The method of claim 13, wherein from 1 mg to 50 mg of eplivanserin or a pharmaceutically acceptable salt of eplivanserin is administered within a 24 hour period.

15. A method for treating cognitive impairment comprising administering eplivanserin or a pharmaceutically acceptable salt of eplivanserin to a subject in need of such treatment, wherein the cognitive impairment is characterized by a deficit in mental activities associated with thinking, reasoning, learning, memory and/or sensory processing and the cognitive impairment is selected from the group consisting of agnosia, amnesia, aphasia, apraxia, delirium, dementia and a learning disorder and the cognitive impairment is associated with a condition selected from the group consisting of AIDS dementia complex, Binswanger's disease, Lewy body dementia, frontotemporal dementia, mild cognitive impairment, multi-infarct dementia, Pick's disease, semantic dementia, Alzheimer's disease, Parkinson's disease, Huntington's disease, Amyotrophic Lateral Sclerosis (ALS), Creutzfeld-Jacob disease, Kuru disease, corticobasal degeneration, frontotemporal lobar degeneration, multiple sclerosis, normal pressure hydrocephalus, organic chronic brain syndrome, Pick's disease, progressive supranuclear palsy, senile dementia, vascular dementia, autism spectrum disorder, Down Syndrome, Fragile X Syndrome, Angelman syndrome, Prader-Willi syndrome, Rett syndrome, childhood disintegrative disorder, attention deficit disorder, attention deficit hyperactivity disorder, obsessive-compulsive disorder, brain trauma, infection, drug intoxication, drug abuse, sleep deprivation, anxiety disorders, dissociative disorders, mood disorders, schizophrenia, somatoform and factitious disorders and chronic pain, wherein the eplivanserin or a pharmaceutically acceptable salt of eplivanserin is administered from one to three times a day and from 1 mg to 50 mg of eplivanserin or a pharmaceutically acceptable salt of eplivanserin is administered within a 24 hour period.

16. A method of treating Lewy-body dementia in a patient diagnosed with a cognitive disorder comprising administering eplivanserin or a pharmaceutically acceptable salt thereof.

17. A method of treating psychosis comprising administering eplivanserin or a pharmaceutically acceptable salt thereof to a patient in need thereof.

18. A method of treating Parkinson's disease psychosis comprising administering eplivanserin or a pharmaceutically acceptable salt thereof to a patient in need thereof.

19. A method of treating Alzheimer's disease psychosis comprising administering eplivanserin or a pharmaceutically acceptable salt thereof to a patient in need thereof.
